(19) United States
(12) Patent Application Publication
(10) Pub. No.: US 2016/0324190 A1
(43) Pub. Date: Nov. 10, 2016

(54) USE OF AN ENZYMATIC COMPOSITION IN THE FEED OF RUMINANTS

(71) Applicant: ETABLISSEMENTS J. SOUFFLET, Nogent Sur Seine (FR)

(72) Inventors: Benoît DELORD, CEYRAT (FR); Mathieu TOURNAT, VANNES (FR)

(21) Appl. No.: 15/108,253

(22) PCT Filed: Jan. 27, 2015
(86) PCT No.: PCT/EP2015/051621

§ 371 Date: Jun. 24, 2016

(30) Foreign Application Priority Data
Jan. 27, 2014 (FR) 1450650

(51) Int. Cl. A23K 20/189; C12N 9/42; C12N 9/24; A23K 50/10; A23K 10/12

(52) U.S. Cl. CPC A23K 20/189; A23K 50/10; A23K 10/12; C12N 9/2482; C12N 9/244; C12N 9/2437; C12Y 302/01004; C12Y 302/01006; C12Y 302/01008

(57) ABSTRACT

The present invention relates to an enzymatic composition comprising a main substrate, selected from the group consisting of wheat, wheat bran, rapeseed cake and a mixture thereof, fermented with a strain of Aspergillus section Nigri, said enzymatic composition having: (i) a xylanase activity greater than or equal to 500 AXC per gram of composition, (ii) a β-glucanase activity of greater than or equal to 500 BGU per gram of composition, and (iii) a cellulase activity greater than or equal to 50 CMC per gram of composition; as well as to its use in the feed of ruminants.

The present invention relates to improvement in the zootechnical performances of ruminants by addition of non-medicinal food additives in the feed.

Genetic progress in ruminants has given the possibility of obtaining livestock animals characterized by very high zootechnical performances. This is expressed by very high milk production levels (more than 40 kg/cow/day) and very high weight gains in cattle for fattening (more than 1,500 g/animal/day). These performances require greater ingestion of foodstuffs by the animals and a very rich feed in order to cover their maintenance and production needs.

The increase in the ingestion level and the increase in the portion of concentrate in the ration of ruminants have the effects of reducing the metabolic efficiency of the animals. The animals use the nutrients much less since transit is accelerated and is therefore less effective. Therefore there exists a significant need for finding nutritional solutions capable of improving the yield of transformation of the ration (fodder and concentrates) into products (milk or meat).

It is known that what limits the overall digestibility of the ration of ruminants is the digestibility of the fibers, cellulose and hemicellulose, whilst the use of enzymes in the feed of ruminants is described in the prior art (Beauchemin et al. (2003) J. Anim. Sci. E.Suppl.2:E37-E47). Moreover the use of bacterial amylases (US 2009/0324571) or fungal amylases (WO 03/068256) has been proposed in order to increase the digestibility of starch in ruminants. Nevertheless, the authors recognize the significant variability in the responses of the animals to supplementation of enzymes and therefore the need to find specific additives for ruminants on the one hand, and on the other hand, the need to find additives targeting improvement in the digestibility of the majority polysaccharides in the feed of ruminants: cellulose and hemicellulose. It was thus proposed to add to the feed of ruminants, purified enzymatic complexes including cellulase, xylanase, β-glucanase, pectinase, mannanase, and α-galactosidase activities in order to increase the digestibility of the ingested fodder (WO 2009/0006362). However, these additives are only used with fodder and cannot be used with concentrated foodstuffs (cereals, byproducts, pressed cakes) while the addition of concentrated foodstuffs is the main means used for improving zootechnical performances of ruminants.

Therefore there exists a significant need for new additives to the feed of ruminants enabling improving the zootechnical performances of the animals, even fed with an optimized feed for example comprising concentrated foodstuffs.

The use of different types of microorganisms which produce enzymes has been proposed in order to improve the zootechnical performances of ruminants (Beauchemin et al. (2004) Can. J. Anim Sci. 84:23-36). Once the fermentation leading to the production of these enzymes is completed, the enzymes are generally separated from the fermentation residues and from the producing microorganisms. However, the types and activities of the produced enzymes widely vary according to the type of microorganism used, to the substrate used and to the culture conditions used, which leads to strong heterogeneity in the effects observed in animals and therefore limits their benefit for the farmer. In particular, the use of an efficient combination of enzymatic activities is difficult to determine, especially with enzymes produced by genetically modified organisms in liquid-state fermentation. The method for administering the enzymes is also a problem when enzymes are used in liquid form, since they are rapidly leached out and inactivated in the rumen.

The present invention results from the unexpected finding by the inventors that it is possible to reproducibly obtain a multi-enzyme composition displaying cellulase, xylanase and β-glucanase enzymatic activities at given levels, by solid-state fermentation of cereals and/or of cereal byproducts and/or of oilseed byproducts with a filamentous fungus of the type Aspergillus section Nigri, more particularly of the Aspergillus tubingensis type, and that this enzymatic composition enables, when it is provided in the feed ration of a ruminant, whatever it is, improving its zootechnical performances, in particular improving milk production, food efficiency, consumption index, gain in weight, increasing rumination activity, increasing the capability of ingesting fodder, increasing the digestibility of the fodder and/or of the concentrates and reducing the production of methane.

The present invention thus relates to an enzymatic composition comprising a main substrate, selected from the group consisting of wheat, wheat bran, rapeseed cake, a mixture of a wheat and wheat bran, a mixture of wheat and rapeseed cake, a mixture of wheat bran and rapeseed cake, and a mixture of wheat, wheat bran and rapeseed cake, fermented with a strain of Aspergillus section Nigri, more particularly of Aspergillus tubingensis, said enzymatic composition displaying:
(i) a xylanase activity of more than or equal to 500 AXC per gram of composition,
(ii) a β-glucanase activity greater than or equal to 500 BGU per gram of composition, and
(iii) a cellulase activity greater than or equal to 50 CMC per gram of composition.

It further concerns a method for manufacturing this enzymatic composition comprising a step of solid-state fermentation of a main substrate, selected from the group consisting of wheat, wheat bran, rapeseed cake, a mixture of wheat and wheat bran, a mixture of wheat and rapeseed cake, a mixture of wheat bran and rapeseed cake, and a mixture of wheat, wheat bran and rapeseed cake, with a strain of sub-clade A. tubingensis, more particularly with a strain of Aspergillus tubingensis or Aspergillus neoniger, more preferably with a strain of Aspergillus tubingensis, at least until the fermentation product displays the following minimal values of enzymatic activity:
(i) a xylanase activity greater than or equal to 500 AXC per gram of composition,
(ii) a β-glucanase activity greater than or equal to 500 BGU per gram of composition, and
(iii) a cellulase activity greater than or equal to 50 CMC per gram of composition, the fermentation being preferably stopped upon appearance of the first spores in the culture medium.

It also relates to an additive for the feed of ruminants which comprises this enzymatic composition.

The object of the present invention is also the use of this additive as an ingredient, in particular as a non-medicinal ingredient, of feed compositions or premix for feeding ruminants.

It also deals with a premix for feeding ruminants comprising this additive, as well as with a supplemented feed composition for ruminants comprising an effective amount of this additive or of this premix, in association with feedstuffs suitable for ruminants.

Another object of the present invention relates to a method for manufacturing a supplemented feed composition for ruminants comprising mixing the additive as defined above or the premix as defined above with feedstuffs suitable for ruminants.

The present invention also relates to the use of the enzymatic composition as defined above, of the additive as defined above, of the premix as defined above or of the feed composition as defined above, for improving the zootechnical performances of a ruminant.

Finally, it is directed to a method for improving the zootechnical performances of a ruminant in which the ruminant is made to ingest an effective amount of the enzymatic composition as defined above, of the additive as defined above, of the premix as defined above or of the feed composition as defined above.

### DETAILED DESCRIPTION OF THE INVENTION

### Ruminants

In the context of the invention, the term [symbol] ruminants [symbol] designates any polygastric herbivorous mammal, for which the digestion takes place totally or partly through a rechewing process of the feed after its ingestion.

The digestive tract of ruminants consists of 4 compartments: the rumen, the reticulum, the omasum and the abomasum. Among these 4 compartments, the rumen is the most important since most nutrients are digested therein. The digestion therein corresponds to a fermentation process involving a significant and varied microbial flora responsible for cellulolysis, hemicellulolysis, proteolysis, production of acid, of methane, synthesis of vitamins, etc. Because of the primordial role of the ruminal flora, the digestive physiology of ruminants is therefore considerably different from that of monogastric animals, for which the flora plays a secondary role.

Such animals are well known to one skilled in the art and for example include bovine animals, ovine animals, caprine animals, cervids and camelids. Preferably, within the background of the invention, the ruminant is a bovine animal.

By [symbol] bovine [symbol] or [symbol] bovine animal [symbol] , is meant here a sub-family of Bovidae comprising several significant species of livestock. Bovine animals in particular include cows, in particular dairy cows, suckler cows, heifers, calves, weanlings, bullocks, raised beef, beef for fattening, bulls, buffalos, yaks, gaurs and bantengs. Preferably, when the ruminant used within the scope of the invention is a bovine animal, it is selected from cows, in particular milk cows, calves, weanlings, milk-fed calves, raised beef and beef for fattening.

By [symbol] ovine [symbol] , are meant here the ruminant herbivores of the Ovis genus. Ovine animals in particular include wild sheep, sheep, ewes, young ewes and lambs.

By [symbol] caprine animal [symbol] , are meant here the ruminant herbivores of the Capra genus. Caprine animals in particular include goats, billy goats, kid goats and ibexes.

By [symbol] cervid [symbol] , are meant here ruminants from the Cervidae family, bearing antlers. Cervids in particular include, stags, fawns, young stags, hinds, fawns, roebucks, bucks, does, reindeer, fallow deer, does (female of fallow deer) and elks.

By [symbol] camelids [symbol] , are meant here artiodactyla mammals of the Camelidae family. Camelids include in particular dromedaries, camels, she-camels, llamas and alpacas.

Preferably, within the context of the invention, the ruminant is a livestock animal.

### Enzymatic Composition

The enzymatic composition according to the invention comprises, essentially consists in, or consists in a main substrate, selected from the group consisting of wheat, wheat bran, rapeseed cake, a mixture of wheat and wheat bran, a mixture of wheat and rapeseed cake, a mixture of wheat bran and rapeseed cake, and a mixture of wheat, wheat bran and rapeseed cake, fermented with a strain of Aspergillus section Nigri, preferably a strain of Aspergillus tubingensis, said enzymatic composition displaying:
(i) a xylanase activity greater than or equal to 500 AXC per gram of composition,
(ii) a β-glucanase activity greater than or equal to 500 BGU per gram of composition, et
(iii) a cellulase activity greater than or equal to 50 CMC per gram of composition.

The enzymatic activities above are expressed per gram of dried composition.

By [symbol] xylanase [symbol] , is meant here an enzyme of the EC 3.2.1.8 class from the nomenclature of enzymes of the International Union of Biochemistry and Molecular Biology (IUBMB), which degrades the linear polysaccharide β-1,4-xylane into xylose, in particular thereby degrading hemicellulose.

Techniques for measuring the xylanase activity are well known to one skilled in the art. Typically, in order to demonstrate this activity, it is possible to make the enzymatic solution act on a solution of soluble oats xylan bound to a chromophore, Rémazol Brillant Blue R, and to measure the oligomers released by the action of the enzymatic solution. The oligomers are found in the soluble fraction after precipitation with ethanol. The reaction medium preferably used in this method consists of 130 μL of oats azo-xylan solution (MEGAZYME) 10 g/L, 50 μL of enzymatic solution diluted in an acetate buffer 0.4 M, pH 4.70. The reaction is preferably conducted at 31° C. for 20 minutes. The reaction is typically stopped by adding 500 μL of 96% ethanol. The optical density of the supernatant obtained after centrifugation (10 minutes at 3,000 rpm at 20° C.) is read out at 590 nm. A xylanase activity unit (AXC) may then be defined as the amount of enzyme which, diluted to 1 unit/mL, at a pH of 4.70 and at 30° C., releases, from a solution of Rémazol Brillant Blue R xylan, oligomers which cannot be precipitated in ethanol such that the optical density of the supernatant is 0.93 at 590 nm.

Preferably, the enzymatic composition according to the invention displays a xylanase activity greater than 500 AXC per gram of composition, more preferably a xylanase activity greater than 750 AXC per gram of composition, more preferably a xylanase activity greater than 1,000 AXC per gram of composition, preferably among all, a xylanase activity greater than or equal to 2,000 AXC per gram of composition.

By [symbol] β-glucanase [symbol] , is meant here an enzyme of the class EC 3.2.1.6 from the nomenclature of enzymes of the International Union of Biochemistry and Molecular Biology (IUBMB), which cleaves glucan. β-glucanases in particular include β-1,3-glucanase which cleaves β-1,3-glucans, such as callose or curdlan, β-1,6-glucanase which cleaves β-1,6-glucans, cellulase, endo-β-1,4-glucanase specific to xyloglucan or exo-β-1,4-glucanase specific of xyloglucan.

Techniques for measuring the β-glucanase activity are well known to one skilled in the art. Typically, for demonstrating this activity, it is possible to make the enzymatic solution react on a solution of barley β-glucan bound to a chromophore, Rémazol Brillant Blue R. Hydrolysis at pH 4.8 of this substrate by the β-glucanase activities releases oligomers bound to the chromophore and which cannot be precipitated with ethanol, the concentration of which is evaluated by measuring the absorbance at 590 nm. The reaction medium preferably used in this method consists of 130 μL of a solution of azo-barley glucan (MEGAZYME) diluted to ⅘^{th} in a 0.1 M acetate phosphate dilution buffer and adjusted to pH 4.75, 50 μL of enzymatic solution diluted in 0.1 M acetate phosphate buffer, pH 4.60. Preferably the reaction is conducted at 31° C. for 20 minutes. The reaction is typically stopped by adding 620 μL of a precipitation solution (30 g of sodium acetate trihydrate and 3 g of zinc acetate in 1 litre of 96% ethanol). The optical density of the supernatant obtained after centrifugation (10 minutes at 3,000 rpm at 20° C.) is read out at 590 nm. A unit of β-glucanase activity (BGU) may then be defined as the amount of enzyme which, diluted to a concentration of 1 unit per mL, under assay conditions (30° C. and pH 4.8), releases oligomers of barley β-glucan bound to Rémazol Brillant Blue R which cannot be precipitated in ethanol, so that the absorbance of the supernatant is 0.9 at 590 nm.

Preferably, the enzymatic composition according to the invention displays a β-glucanase activity greater than 500 BGU per gram of composition, more preferably a β-glucanase activity greater than 600 BGU per gram of composition, more preferably a β-glucanase activity greater than 1,000 BGU per gram of composition, preferably among all, a β-glucanase activity greater than or equal to 1,500 BGU per gram of composition.

By [symbol] cellulase [symbol] , is meant here an enzyme from the class EC 3.2.1.4 of the nomenclature of enzymes of the International Union of Biochemistry and Molecular Biology (IUBMB), which cleaves cellulose, lichenin and β-glucans of cereals, polymer of glucoses bound in β1-4. The [symbol] cellulases [symbol] in particular include endo-1,4-β-D-glucanase, β-1,4-glucanase, β-1,4-endoglucane hydrolase, cellulase A, cellulosin AP, endoglucanase D, alkali cellulase, cellulase A 3, celludextrinase, 9.5 cellulase, avicelase, pancellase SS and 1,4-(1,3,1,4)-β-D-glucane 4-glucanohydrolase.

Techniques for measuring cellulase activity are well known to one skilled in the art. Typically, in order to demonstrate this activity, it is possible to make the enzymatic solution act on a solution of carboxymethyl cellulose partly depolymerized and bound to a chromophore, Rémazol Brillant Blue R. Hydrolysis at pH 4.5 of this substrate by the cellulase activities releases oligomers bound to the chromophore and which cannot be precipitated with ethanol, the concentration of which is evaluated by measuring the absorbance at 590 nm. The reaction medium preferably used in this method consist of 100 μL of azo-carboxymethyl-cellulose solution (Megazyme 90504a) at 20 g/L and pH 4.5, 100 μL of enzymatic solution diluted in 0.1 M acetate buffer, pH 4.60. The reaction is preferably conducted at 41° C. for 10 minutes. The reaction is typically stopped by adding 500 μL of a precipitation solution (40 g of sodium acetate trihydrate and 4 g of zinc acetate in 1 litre of 96% ethanol). The optical density of the supernatant obtained after centrifugation (10 minutes at 3,000 rpm at 20° C.) is read out at 590 nm. A unit of carboxymethylcellulase activity (CMC) may then be defined as the amount of enzyme which, diluted to a concentration of 1 unit per mL under assay conditions (41° C. and pH 4.5), releases oligomers of carboxymethyl cellulose partly depolymerized bound to Rémazol Brillant Blue R which cannot be precipitated with the precipitation solution, so that the absorbance of the supernatant is 1.0 at 590 nm.

Preferably, the enzymatic composition according to the invention displays a cellulase activity greater than 50 CMC per gram of composition, more preferably a cellulase activity greater than 75 CMC per gram of composition, more preferably a cellulase activity greater than 120 CMC per gram of composition, preferably among all, a cellulase activity greater than or equal to 180 CMC per gram of composition.

In a particularly preferred embodiment, the enzymatic composition according to the invention displays a xylanase activity greater than or equal to 2,000 AXC per gram of composition, a β-glucanase activity greater than or equal to 1,500 BGU per gram of composition and a cellulase activity greater than or equal to 180 CMC per gram of composition.

The enzymatic composition according to the invention may further comprise enzymatic activities other than the xylanase, β-glucanase and cellulase activities above, but at very low levels. Preferably, the enzymatic composition according to the invention is devoid of any pectinase, mannanase, amylase and/or α-galactosidase activity. Preferably, the enzymatic composition according to the invention is devoid of any ferulic acid esterase activity.

By [symbol] composition devoid of any enzymatic activity X [symbol] , is meant here a composition in which the enzymatic activity X is not detectable with conventional techniques for detecting enzymatic activity. In other words, a composition devoid of any enzymatic activity X does not comprise any protein having an enzymatic activity X or comprises proteins having an enzymatic activity X in a so low concentration (for example, determined by proteomic techniques) that the corresponding enzymatic activity X cannot be measured with conventional techniques for detecting enzymatic activity.

The xylanase, β-glucanase and cellulase activities above originatet from the fermentation of the substrate, in particular of the main substrate, comprised in the enzymatic composition according to the invention, with a strain of Aspergillus section Nigri, more particularly a strain of Aspergillus tubingensis.

By [symbol] main substrate [symbol] , is meant here the substrate which represents at least 70%, preferably at least 75%, at least 80%, at least 85%, at least 90%, at least 95% or 100% of the substrate used during the fermentation with a strain of Aspergillus section Nigri, more particularly a strain of Aspergillus tubingensis.

This substrate, in particular this main substrate, is selected from the group consisting of wheat, wheat bran, rapeseed cake, a mixture of wheat and wheat bran, a mixture of wheat and rapeseed cake, a mixture of wheat bran and rapeseed cake, and a mixture of wheat, wheat bran and rapeseed cake. Preferably, the substrate is selected from the group consisting of wheat bran, rapeseed cake, and a mixture thereof. Preferably among all, the substrate, in particular the main substrate, is a mixture of wheat bran and rapeseed cake. The wheat bran and the rapeseed cake may be present in the mixture in mass proportions ranging from 10/90 (in other words 10% of wheat bran for 90% of rapeseed cake) to 90/10 (in other words 90% of wheat bran for 10% of rapeseed cake), preferably 15/85 (in other words 15% of wheat bran for 85% of rapeseed cake) to 80/20 (in other words 80% of wheat bran for 20% of rapeseed cake), preferably from 20/80 (in other words 20% of wheat bran for 80% of rapeseed cake) to 70/30 (in other words 70% of wheat bran for 30% of rapeseed cake), preferably from 25/75 (in other words 25% of wheat bran for 75% of rapeseed cake) to 60/40 (in other words 60% of wheat bran for 40% of rapeseed cake), still more preferably from 20/80 (in other words 20% of wheat bran for 80% of rapeseed cake) to 50/50 (in other words 50% of wheat bran for 50% of rapeseed cake), still more preferably from 30/70 (in other words 30% of wheat bran for 70% of rapeseed cake) to 50/50 (in other words 50% of wheat bran for 50% of rapeseed cake).

The substrate used during the fermentation with a strain of Aspergillus section Nigri, more particularly a strain of Aspergillus tubingensis, may further comprise as an additional substrate:
oilseed cakes, such as soybean cakes, maize germ cakes, sunflower cakes, palmist cakes, flax cakes, groundnut cakes, cocoa cakes, coprah cakes, cotton cakes, grape pip cakes, olive pomace, cameline cakes, jatropha seed cakes or sesame cakes;
cereal byproducts other than wheat bran, such as flour, sharps, brown remolding, bran, gluten feed, gluten meal, dried grains from distilleries/ethanol plants, dried and soluble grains from distilleries/ethanol plants, brewer's grains, ginned ears of corn, rootlets, germs or meal, of oats, wheat, barley, triticale, rice, rye or maize;
organic nitrogen sources such as amino acids, concentrates of soya proteins, concentrates of potato proteins, wheat gluten or maize gluten;
inorganic nitrogen sources such as urea, ammonia, ammonium salts or stillage;
sodium, potassium, chlorine, calcium or phosphorus mineral salts; and/or
trace elements such as iron, copper, zinc, manganese, cobalt, iodine or selenium.

Preferably, the substrate used during the fermentation with a strain of Aspergillus section Nigri, more particularly a strain of Aspergillus tubingensis, may further comprise as an additional substrate oilseed cakes, in particular maize germ cakes.

The substrate as defined above is fermented with a strain of Aspergillus section Nigri, preferably a strain of Aspergillus section Nigri clade A. niger, more preferably a strain of Aspergillus tubingensis or Aspergillus neoniger, more preferably a strain of Aspergillus tubingensis, in order to obtain the xylanase, β-glucanase and cellulase activities as defined above. In an alternative embodiment, the substrate as defined above is fermented with a strain of Aspergillus section Nigri which is not a strain of Aspergillus tubingensis. The fermentation may be an immersed fermentation (or a liquid-state fermentation), a solid-state fermentation or a solid/liquid-state fermentation. Preferably, the fermentation is a solid-state fermentation.

In the context of the invention, [symbol] solid-state fermentation [symbol] is defined as the culture of microorganisms on humid solid substrates, on inert substrates or on insoluble substrates which can be used as a source of carbon and energy. The fermentation process takes place in the absence or quasi-absence of free water in the space between the substrate particles. On the contrary, [symbol] immersed fermentation [symbol] here refers to a culture of microorganisms in which, both the nutrients and the microorganisms are immersed in an aqueous medium.

Preferably, the substrate as defined above is fermented by solid-state fermentation with a strain of Aspergillus tubingensis.

By [symbol] Aspergillus section Nigri [symbol] is meant here a section of the Aspergillus genus previously designated as group A. niger, which typically comprises 26 species as described in Varga et al. (2011) Studies in Mycology 69:1-17, grouped in 5 main clades: the clade A. niger, which comprises the species A. neoniger, A. costaricaensis, A. vadensis, A. eucalypticola, A. piperis, A acidus, A. tubingensis, A. awamori, A. niger and A. brasiliensis; the clade A. carbonarius, which comprises the species A. ibericus, A. sclerotiicarbonarius, A carbonarius and A. sclerotioniger; the clade A. heteromorphus, which comprises the species A. ellipticus and A. heteromorphus; the clade A. homomorphus, which comprises the species A. homomorphus; and the clade A. aculeatus, which comprises the species A. fijiensis, A. aculeatus, A. aculeatinus, A. uvarum, A. indologenus, A. japonicus and A. violaceofuscus. Preferably, the strain of Aspergillus section Nigri used within the scope of the invention is a strain of the clade A. niger. In a particular embodiment, the strain of Aspergillus section Nigri used within the scope of the invention is a strain of the clade A. niger which is not a strain of A. tubingensis.

As indicated above, the clade A. niger comprises 10 species grouped in 3 sub-clades: the sub-clade A. tubingensis, which comprises the species A. neoniger, A. costaricaensis, A. vadensis, A. eucalypticola, A. piperis, A acidus and A. tubingensis; the sub-clade A. niger, which comprises the species A. awamori and A. niger; and the sub-clade A. brasiliensis which comprises the species A. brasiliensis. Preferably, the strain of Aspergillus section Nigri used within the scope of the invention is a strain of the sub-clade A. tubingensis. In a particular embodiment of the invention, the strain of Aspergillus section Nigri used within the scope of the invention is a strain of the sub-clade A. tubingensis which is not a strain of A. tubingensis. More preferably, the strain of Aspergillus section Nigri used within the scope of the invention is a strain of Aspergillus tubingensis or Aspergillus neoniger, even more preferably a strain of Aspergillus tubingensis.

By [symbol] Aspergillus tubingensis [symbol] , is meant here a member of the Aspergillus section nigri, the characteristic strain of which is the strain Aspergillus tubingensis Mosseray described in La Cellule (1934) 43:245-247. It is a filamentous fungus producing black conidia, ubiquitous of the ground, saprophytic and capable of growing on many complex natural substrates. Aspergillus tubingensis does not appear in the list of pathogenic agents of annex III of the Directive 2000/54/EC relating to the protection of workers against the risks related to biological agents at work.

Examples of strains of Aspergillus tubingensis are well known to one skilled in the art and include Aspergillus tubingensis Mosseray ATCC MYA-81, Aspergillus tubingensis Mosseray ATCC MYA-83, Aspergillus tubingensis Mosseray ATCC MYA-84, Aspergillus tubingensis Mosseray ATCC MYA-4879, Aspergillus tubingensis Mosseray ATCC MYA-77, Aspergillus tubingensis Mosseray ATCC MYA-78, Aspergillus tubingensis Mosseray ATCC MYA-79, Aspergillus tubingensis Mosseray ATCC MYA-82, Aspergillus tubingensis Mosseray ATCC MYA-80, Aspergillus tubingensis Mosseray ATCC 10550, Aspergillus tubingensis Mosseray ATCC 76608 and Aspergillus tubingensis Mosseray ATCC 201255.

By “Aspergillus neoniger”, is meant here a member of the Aspergillus section nigri, for which the characteristic strain is the strain Aspergillus neoniger Varga described in Varga et al. (2011) Studies in Mycology 69:1-17.

Examples of strains of Aspergillus neoniger are well known to one skilled in the art and include Aspergillus neoniger CBS 115656 or NRRL 62634 and Aspergillus neoniger CBS 115657.

Preferably, the substrate as defined above is pre-treated before fermentation in order to be pasteurized or sterilized. The heat treatment may consist in heating in an autoclave for example. The substrate may thus be autoclaved for 15 to 45 min, preferably for 20 to 40 min, more preferably for 35 min, at a temperature comprised between 90 and 125° C., preferably between 95 and 115° C., more preferably at a temperature of 105° C.

More preferably, the substrate as defined above is pre-humidified so as to attain 40 to 100% of dry matter, preferably 45 to 90% of dry matter, preferably from 50 to 80% of dry matter, preferably from 55 to 70% of dry matter, preferably 60% of dry matter.

Advantageously, it is possible to adjust the pH during the humidification in the range from 4.8 to 6.2, preferably from 5.0 to 6.0, preferably from 5.2 to 5.8, preferably among all to 5.6, in order to improve the pasteurizing effect of the heat treatment and the starting of the desired fermentation.

The inoculation of the substrate as defined above may be carried out with any suitable inoculum. One skilled in the art is aware of multiple ways for preparing a suitable inoculum from a selected strain of Aspergillus tubingensis. The inoculation dose is advantageously at least 1×10⁷ spores per gram of initial dry matter of substrate.

The water content of the substrate at the beginning of the fermentation is preferably adjusted between 40 and 50%, preferably to 45% of the total mass of the substrate and of the water and is preferably maintained substantially in this interval during fermentation, for example by periodically proceeding with additions of water for compensating the loss of water of the medium. The expression “substantially maintained” means that it is tolerable that the humidity content has a value away from 5 units % from the interval 40-50% during a relatively short period between two successive adjustments of the humidity level or at the end of the fermentation. The humidity level of the substrate may indeed tend to decrease during fermentation by evaporation under the effect of the increase of the temperature generated by the fungal growth.

The fermentation may be conducted in any suitable reactor. Examples of reactors which may be used are those described in the article of A. Durand et al. published in Agro-Food-Industry Hi-Tech (Mai-Juin 1997, pages 39-42).

The fermentation may be conducted for a period from 1 to 3 days, preferably from 30 to 60 hours, more preferably for 48 hours. Preferably, the fermentation is stopped upon appearance of the first spores in the culture medium, because the presence of spores may bother the animals upon ingestion because of their volatile nature.

The temperature of the medium is preferably maintained between 28 and 38° C., preferably between 30 and 36° C., more preferably to 33° C.

Preferably, the fermentation is carried out under aerobic conditions and preferably in darkness.

The thereby obtained fermentation product is a humid solid product. It may be dried or dehydrated, preferably at a moderate temperature, for example of less than 45° C., so as not to affect the enzymatic activity. The drying or dehydration may be carried out with any suitable technique well known to one skilled in the art, such as the use of a fluidized bed, freeze-drying, steaming, steaming in vacuo or zeodration.

It may also be frozen, preferably in the humid state, at a low temperature, for example −20° C.

The fermentation product may also be extracted in water and recovered in liquid form by any suitable technique well known to one skilled in the art. The extraction of the fermentation product may be performed by any technique well known to one skilled in the art, in particular by extraction in an aqueous solution, extraction in an alcoholic solution, extraction with solvents, high pressure homogeneization, supercritical extraction, extraction with fluidized beds, milling, cryogenic milling, decompression, cavitation, bubbling, extraction with ultrasound, adsorption on resins or zeolites. The enzyme(s) in liquid form may then be purified by any technique known to one skilled in the art, in particular by centrifugation, filtration, ultrafiltration, chromatography, use of membranes or precipitation.

The enzymatic composition according to the invention may be in any suitable form for its use in an additive. It is preferably in a crude form, non-extracted.

By [symbol] crude form [symbol] , is meant here the fermentation medium containing the enzymatic activities as defined above, the fermented substrate and the fungus Aspergillus section Nigri, in particular the fungus Aspergillus tubingensis as defined above. The fermentation medium after fermentation of the substrate by a strain of Aspergillus section Nigri, more particularly a strain of Aspergillus tubingensis, may in particular be dehydrated and/or milled before being used directly in the enzymatic composition according to the invention. This milling may be carried out by any suitable technique, well known to one skilled in the art, such as micronization, milling with shearing, milling by impact, cryomilling or crumbling.

The enzymatic composition according to the invention may further be standardized. Such a standardization advantageously gives the possibility of ensuring good homogeneity of the composition and of facilitating its use during the step of manufacturing the feedstuffs.

### Additive for Feeding Ruminants

The present invention relates to an additive, preferably a non-medicinal additive, for the feed for ruminants, as defined in the section [symbol] Ruminants [symbol] above, which comprises, essentially consists in, or consists in an enzymatic composition as defined in the section [symbol] Enzymatic composition [symbol] above.

By [symbol] additive [symbol] , is meant here a component or mixture of components which may be added to a feedstuff, to a feed ration or to a feed diet of an animal, or given to be ingested by the animal.

The additive according to the invention may further comprise additional ingredients such as physiologically acceptable carriers, stabilizers, antioxidants, or preservatives, as well as additional enzymes such as proteases, phytases, mannanases, amylases, alpha-galactosidases, ferulic acid esterases and/or pectinases.

The additive according to the invention may be in any form suitable for its subsequent use, in particular in a liquid, powder or granule form.

The additive according to the invention may be used as an ingredient, in particular as a non-medicinal ingredient, of feed compositions or premixes for feeding ruminants as defined in the section [symbol] Ruminants [symbol] above.

### Premix

By [symbol] premix [symbol] , is meant here a concentrate of enzymes and optionally of trace elements, vitamins and minerals, associated in a low percentage with the different raw materials for making up the complete feed intended for ruminants.

The premix is preferably made up from a dilution or deposition on a support of the additive as defined in the section [symbol] Additive [symbol] above, in order to standardize the enzymatic activity and to facilitate its use in the target animals.

The present invention thus relates to a premix for feeding ruminants as defined in the section [symbol] Ruminants [symbol] above, comprising an additive as defined in the section [symbol] Additive [symbol] above.

The premix according to the invention may comprise other additives conventionally used in the feed of animals, in particular of ruminants.

Such additives are well known to one skilled in the art and include technological additives such as preservatives, antioxidants, emulsifiers, stabilizers, thickeners, gelling agents, binders, substances for controlling contamination of radionuclides, anti-agglomeration agents, acidity correctors, additives for silage, denaturating agents; sensorial additives such as coloring agents (doping or modifying the color of feed for animals, substances which, included in the feed of the animals will give color to foodstuffs produced from these animals), aromatic substances; nutritional additives, such as vitamins, provitamins, omega 3 fatty acids, and [symbol] chemically well defined substances with a similar effect [symbol] , compounds of trace elements, amino acids, urea; fatty acids and zootechnical additives such as digestibility improvement agents like enzymes, stabilizers of intestinal flora, or substances having a [symbol] positive effect on the environment [symbol] .

As an example, the premix according to the invention may have the following composition:

| | |
|---|---|
| Enzymatic composition according to the invention | 0.3-3% |
| Mixture of vitamins (A, B, D, E, nicotinic acid, etc.) | 0.1-1% |
| Mineral salts (CaHPO₄, CaCO₃, NaCl, | 0-99.6% |
| CuO, MnO, FeSO₄, ZnO, etc.) | |
| Substances from cereals (bran, remolding, fodder flour, | 0-99.6% |
| corncob, barley rootlets, etc.) | |

The premix according to the invention may be in any form adapted for its subsequent use, in particular in liquid, powder or granule form.

### Feed Composition

The present invention also relates to a supplemented feed composition for ruminants as defined in the section [symbol] Ruminants [symbol] above, comprising an effective amount of a additive as defined in the section [symbol] Additive [symbol] above or of a premix as defined in the section [symbol] Premix [symbol] above, in association with feedstuffs suitable for ruminants as defined in the section [symbol] Ruminants [symbol] above.

The feedstuffs suitable for ruminants are well known to one skilled in the art and for example include fodders of any types and in all their forms (greens, dehydrated, silage, agglomerates, etc.) like grass and the other fodder grasses, fodder cereals (barley, maize, oats, wheat, sorgho, soya, rye), legumes (peas, faba bean, lupin, soya, lucerne, sainfoin, clovers), roots, tubers and their byproducts (beets, beets pulp, potato, potato pulp, etc.), cabbage, rapeseed, sunflower, vegetable wastes (tops, stalks, cereal husks, bran, husked corncobs, bagasse) and starches, byproducts of the agri-food industry (starch manufacturing, starch production, ethanol plants, breweries, milling, etc), as well as oilseed cakes, syrups, and nitrogen-containing foodstuff materials such as urea and derivatives thereof (biuret, ureides) and ammonia salts.

Preferably, the feedstuffs suitable for ruminants used within the scope of the invention comprise fodder, preferably maize silage typically representing from 10 to 50% of the ingested dry matter, preferably daily, preferably associated with other fodder such as hay, straw or grass or cereal silage and preferably supplemented with concentrated feedstuffs such as cereals, oilseed cakes or composed feedstuffs.

A feed ration for ruminants may notably contain the enzymatic composition as defined in the section [symbol] Enzymatic composition [symbol] above with:
humid, dried or dry fodder in proportions ranging from 0 to 100%, preferably from 40 to 70%.
concentrated feedstuffs (concentrated raw material or composed feedstuff) in proportions ranging from 0 to 100%, preferably from 30 to 60%.

Preferably, the enzymatic composition as defined in the section [symbol] Enzymatic composition [symbol] above, the additive as defined in the section [symbol] Additive [symbol] above or the premix as defined in the section [symbol] Premix [symbol] above is present in the feed composition in an amount such that the effective daily dose, as defined in the section [symbol] Effective daily dose [symbol] below is provided to the animal.

The feed composition according to the invention may be in any suitable form for feeding ruminants, in particular livestock ruminants. In particular it may be in the form of flakes, granules, crumbs or flour.

The present invention also relates to a method for manufacturing a supplemented feed composition for ruminants as defined above comprising a step of mixing an additive as defined in the section [symbol] Additive [symbol] above or a premix as defined in the section [symbol] Premix [symbol] above with feedstuffs suitable for ruminants, in particular livestock ruminants, as defined above.

The step of mixing the additive or the premix with the feedstuff may be carried out by any technique well known to one skilled in the art.

The method for manufacturing the feed composition according to the invention may further comprise a step of formulating the feed composition as well as a conditioning step. These steps may be carried out by any conventional technique well known to one skilled in the art. Thus, the additive or the premix, when they are in the form of powder, may be added to the feedstuff at the moment of the formulation (or mixing) step, or when they are in liquid form, may be sprayed after granulation of the feed.

### Effective Daily Dose

The additive as defined in the section [symbol] Additive [symbol] above, the premix as defined in the section [symbol] Premix [symbol] above, or the feed composition as defined in the section [symbol] Feed composition [symbol] above, are formulated so as to provide the effective amount of enzymatic activities to the target animals. The formulation of the additive, of the premix or of the feed composition may be carried out with any technique well known to one skilled in the art, such as notably mixing, dilution, deposition on a carrier, spraying or powdering on granules, or direct distribution to the animals.

The adopted formulation depends on the target animal and is preferably driven by the level of β-glucanase activity.

The effective daily doses are typically:
milk cows: from 2,250 to 9,000 BGU/animal/day, preferably 4,500 BGU/animal/day
bovine animals for fattening: from 2,250 to 9,000 BGU/animal/day, preferably 2,250 BGU/animal/day up to a live weight of 500 kg and 3,375 BGU/animal/day beyond a live weight of 500 kg.

The effective daily doses for the other types of ruminants may be calculated by one skilled in the art by comparison with milk cows or bovine animals for fattening, according to techniques well known to one skilled in the art, such as notably depending on the live weight, depending on the metabolic live weight (PV^{0.75}), depending on the volume of the rumen, depending on the total amount of dry matter ingested per day, with a minimum amount preferably of 1,125 BGU/animal/day, particularly in animals for which the live weight is less than 100 kg.

### Uses for Increasing the Zootechnical Performances

The inventors showed that supplementing the feed ration of milk cows or bovine animals for fattening with the enzymatic composition according to the invention surprisingly enabled increasing their zootechnical performances, in particular improving milk production, including the butyrous level and the protein level of the produced milk, increasing the weight gain, increasing the rumination activity, improving the food efficiency and the consumption index, increasing the capability of ingestion of fodder, increasing the digestibility of fodder and/or of the concentrates and reducing the production of methane.

The object of the present invention is therefore also the use of an enzymatic composition as defined in the section [symbol] Enzymatic composition [symbol] above, of an additive as defined in the section [symbol] Additive [symbol] above, of a premix as defined in the section [symbol] Premix [symbol] above or of a feed composition as defined in the section [symbol] Feed composition [symbol] above, for increasing the zootechnical performances of a ruminant as defined in the section [symbol] Ruminants [symbol] above.

By [symbol] zootechnical performance [symbol] , is meant here an indicator enabling estimating the quality of an animal, in particular its biological capability for various functions (growth, work, reproduction . . . ). Zootechnical performances in particular include the weight gain of the animal, in particular the daily average weight gain, its consumption index or its food efficiency, the weight at a typical age, the carcass yield, the carcass weight, the carcass conformation, the food intake, the capability of ingesting fodder, the digestibility of the fodders and/or of the concentrates, the milk production, the protein level of the milk, the fats level of the milk, the rumination activity, the production of methane, the litter size or the hairs production. Preferably, the enzymatic composition as defined in the section [symbol] Enzymatic composition [symbol] above, the additive as defined in the section [symbol] Additive [symbol] above, the premix as defined in the section “Premix” above or the feed composition as defined in the section [symbol] Feed composition [symbol] above enables promoting the weight gain and/or improving the consumption index and/or promoting the food intake and/or improving the carcass yield, the carcass weight, the carcass conformation, the milk production, the protein level of the milk, the fats level of the milk, the litter size and/or the production of hairs and/or increasing the rumination activity and/or increasing the capability of ingesting fodders and/or increasing the digestibility of the fodders and/or of the concentrates and/or reducing the production of methane in a ruminant as defined in the section [symbol] Ruminants [symbol] above. More preferably, the enzymatic composition as defined in the section [symbol] Enzymatic composition [symbol] above, the additive as defined in the section [symbol] Additive [symbol] above, the premix as defined in the section [symbol] Premix [symbol] above or the feed composition as defined in the section [symbol] Feed composition [symbol] above enablesf increasing the milk production and/or improving the consumption index and/or promoting the weight gain and/or increasing the rumination activity and/or increasing the capability of ingesting fodders and/or increasing the digestibility of fodders and/or of concentrates and/or reducing the production of methane in a ruminant as defined in the section [symbol] Ruminants [symbol] above. Preferably among all, the enzymatic composition as defined in the section [symbol] Enzymatic composition [symbol] above, the additive as defined in the section [symbol] Additive [symbol] above, the premix as defined in the section [symbol] Premix [symbol] above and the feed composition as defined in the section [symbol] Feed composition [symbol] above enables increasing the milk production and/or improving the consumption index and/or promoting the weight gain and/or increasing the rumination activity in a ruminant as defined in the section [symbol] Ruminants [symbol] above

By [symbol] weight gain [symbol] or [symbol] daily average gain [symbol] , is meant here the evolution in the weight growth of the animal, expressed in g/day.

By [symbol] consumption index [symbol] , is meant here the efficiency of the food conversion. This index is determined by the ratio between the amount of consumed feedstuffs and the live weight gain in the case of ruminants for fattening, and by the ratio between the amount of consumed feedstuffs and the milk production in the case of milk-producing ruminants.

By [symbol] food efficiency [symbol] , is meant here the measurement of the transformation of the feed ration of the ruminants into products. This is the inverse ratio of the consumption index as defined above.

By [symbol] weight at a typical age [symbol] , is meant here the weight of the animal at a reference age (1 year or 18 months for example), which enables facilitating comparisons.

By [symbol] carcass yield [symbol] , is meant here the ratio between the weight of the carcass and the live weight of the animal, which enables estimating the [symbol] true yield [symbol] .

By [symbol] carcass weight [symbol] , is meant here the weight of the carcass after chilling.

By [symbol] food intake [symbol] , is meant the amount of feedstuffs ingested by the animal.

By [symbol] capability of ingesting fodders [symbol] , is meant the amount of fodders spontaneously ingested by the animal.

By [symbol] digestibility of the fodders and/or of the concentrates [symbol] , is meant the share of fodders and/or concentrates effectively retained or digested by the animal.

By [symbol] production of milk [symbol] or [symbol] milk production [symbol] , is meant the amount of milk produced per day, expressed in a unit of volume or a unit of weight. Within the context of the invention, the improvement in milk production may be expressed by an increase in the amount of produced milk, but also by an improvement of the butyrous level and/or of the protein level of the milk.

By [symbol] protein level of the milk [symbol] , is meant the amount of protein matter contained in the milk.

By [symbol] fats level of the milk [symbol] , is meant here the amount of fats contained in the milk.

By [symbol] litter size [symbol] , is meant the number and the weight at birth of the young or offspring.

By [symbol] rumination activity [symbol] , is meant here the number of minutes per day dedicated to chewing the food bolus.

By [symbol] production of hairs [symbol] , is meant the amount of hairs produced by a mammal bred for its hairs (for example a cashmere goat).

Preferably, the enzymatic composition as defined in the section [symbol] Enzymatic composition [symbol] above, the additive as defined in the section [symbol] Additive [symbol] above, the premix as defined in the section [symbol] Premix [symbol] above or the feed composition as defined in the section [symbol] Feed composition [symbol] above enables improving milk production, in particular improving the butyrous level and/or the protein level of the milk, and/or improving the consumption index and/or the food efficiency and/or promoting the weight gain and/or improving the rumination activity and/or increasing the capability of ingesting fodders and/or increasing the digestibility of the fodders and/or of the concentrates and/or reducing the production of methane in a significant way in a ruminant as defined in the section [symbol] Ruminants [symbol] above.

In particular, the enzymatic composition as defined in the section [symbol] Enzymatic composition [symbol] above, the additive as defined in the section [symbol] Additive [symbol] above, the premix as defined in the section [symbol] Premix [symbol] above or the feed composition as defined in the section [symbol] Feed composition [symbol] above enables improving the milk production by 1 to 5%, preferably 3%, and/or improving the protein level of the milk by 0.5 to 1.5%, preferably 1%, in a ruminant, in particular in a bovine animal, preferably in a milk cow, preferably fed with a feed comprising maize silage, preferably a feed comprising rough fodder such as straw or hay, oilseed cakes, beets pulp and nitrogen-containing and energetic concentrates, the enzymatic composition as defined in the section [symbol] Enzymatic composition [symbol] above preferably being present in the feed so as to provide 4,500 BGU/animal/day.

In particular, the enzymatic composition as defined in the section [symbol] Enzymatic composition [symbol] above, the additive as defined in the section [symbol] Additive [symbol] above, the premix as defined in the section [symbol] Premix [symbol] above or the feed composition as defined in the section [symbol] Feed composition [symbol] above also enables improving the weight gain by 1 to 15%, preferably by 10%, in a ruminant, in particular in a bovine animal, preferably in a bovine animal for fattening, preferably fed with a feedstuff comprising maize silage, preferably a feedstuff comprising maize silage and nitrogen-containing and energetic concentrates, the enzymatic composition as defined in the section “Enzymatic composition” above preferably being present in the feed so as to provide 2,250 BGU/anima/day up to a live weight of 500 kg and 3,375 BGU/animal/day beyond a live weight of 500 kg.

In particular, the enzymatic composition as defined in the section [symbol] Enzymatic composition [symbol] above, the additive as defined in the section [symbol] Additive [symbol] above, the premix as defined in the section [symbol] Premix [symbol] above or the feed composition as defined in the section [symbol] Feed composition [symbol] above also enables increasing the rumination activity by 5 to 15%, preferably from 8 to 11%, in a ruminant, in particular in a bovine animal, preferably in a milk cow, preferably fed with a feedstuff comprising maize silage, preferably a feedstuff comprising maize silage and nitrogen-containing and energetic concentrates, the enzymatic composition as defined in the section [symbol] Enzymatic composition [symbol] above preferably being present in the feed so as to provide 4,500 BGU/animal/day.

In particular, the enzymatic composition as defined in the section [symbol] Enzymatic composition [symbol] above, the additive as defined in the section [symbol] Additive [symbol] above, the premix as defined in the section [symbol] Premix [symbol] above or the feed composition as defined in the section [symbol] Feed composition [symbol] above also enables reducing the production of methane by 0.5 to 9%, preferably by 2.5 to 8%, in a ruminant, in particular in a bovine animal, preferably in a culled cow, preferably fed with a feedstuff comprising grass hay, maize silage and/or grass silage, preferably a feedstuff comprising grass hay, the enzymatic composition as defined in the section [symbol] Enzymatic composition [symbol] above preferably being present in the feed so as to provide 3,375 BGU/animal/day.

The enzymatic composition as defined in the section [symbol] Enzymatic composition [symbol] above, the additive as defined in the section [symbol] Additive [symbol] above, the premix as defined in the section [symbol] Premix [symbol] above and the feed composition as defined in the section [symbol] Feed composition [symbol] above, further enable maintaining a good general condition of the ruminant or of the herd of ruminants to which they are administered, and in particular maintaining fertility, morbidity and/or mortality of the herd which are acceptable for the farmer, or even promoting this fertility and/or reducing this morbidity and/or this mortality.

The present invention also relates to a method for increasing the zootechnical performances of a ruminant characterized in that the ruminant is made to ingest, preferably in the feed ration, an effective amount of the enzymatic composition as defined in the section [symbol] Enzymatic composition [symbol] above, of the additive as defined in the section [symbol] Additive [symbol] above, of the premix as defined in the section [symbol] Premix [symbol] above or of the feed composition as defined in the section [symbol] Feed composition [symbol] above.

By [symbol] effective amount [symbol] , is meant here the amount of enzymatic composition, optionally present in the additive, the premix or the feed composition, enabling increasing the zootechnical performances as defined above. As this will be clearly apparent for one skilled in the art, this amount will depend on the relevant ruminant, on its age, on its gender, on its genetic type, on its physiological stage, on its weight, on its expected performance level and on its feed ration.

Typically, in the case of bovine animals, the effective ingested amount is such that the ingested β-glucanase activity is comprised between 2,250 and 9,000 BGU/day, preferably 4,500 BGU/day for a milk cow, and between 2,250 to 9,000 BGU/day for a bovine animal for fattening, preferably 2,250 BGU/day for a bovine animal for fattening having a live weight up to 500 kg and 3,375 BGU/day for a bovine animal for fattening having a live weight above 500 kg.

Generally, the enzymatic composition as defined in the section [symbol] Enzymatic composition [symbol] above is provided to the ruminant in the form of successive intakes spread out in time, for example according to daily rhythms, biweekly, weekly or else bimonthly rhythms. Preferably, the ruminants are made to ingest an effective amount of enzymatic composition as defined in the section [symbol] Enzymatic composition [symbol] above according to a daily dose.

For the ruminants which are exclusively fed by grazing, the enzymatic composition as defined in the section [symbol] Enzymatic composition [symbol] above will preferably be provided separately from their main feed.

For the ruminants which are fed with fresh fodder or with stored fodder (e.g. hay), or else with industrial feedstuffs, including with feed concentrates, the enzymatic composition as defined in the section [symbol] Enzymatic composition [symbol] above may be provided in a mixture with these feedstuffs or in a separate form of these feedstuffs.

The present invention will be illustrated in more detail with the examples below.

### EXAMPLES

### Example 1

### Production of the Enzymatic Composition According to the Invention by Solid-State Fermentation

In this example us described a method for obtaining the enzymatic composition according to the invention by solid-state fermentation of a rapeseed cake and wheat bran mixture in the presence of a strain of Aspergillus tubingensis.

A nutritive medium is formed with a mixture of rapeseed cake as a flour and wheat bran in a proportion of 7/3 by weight. The mixture is then pre-humidified to 60% of dry matter and autoclaved for 35 min at 105° C. After cooling, the medium is inoculated with a solution of spores of Aspergillus tubingensis in order to obtain a concentration of 1×10⁷ spores per gram of dry matter and an initial humidity of 45%. The pH is adjusted to 4.9 by adding sulfuric acid. The thereby obtained culture medium is distributed in Erlenmeyer vials in an amount of 10 g of dry matter per vial. The Erlenmeyer vials are then incubated at 33° C. under aerobic conditions in darkness for 48 h without any stirring. The culture is stopped upon occurrence of the first spores in the culture medium (since the presence of spores may bother the animals during the ingestion because of their volatility, the fermented products are designed so as to contain a minimum possible amount of spores).

The mixture obtained at the end of the fermentation may be dried and used as such in the feeding of the animals or be extracted in water so as to be added in liquid form.

### Example 2

### Measurement of the Effect of the Enzymatic Composition According to the Invention on the Production Performances of Milk Cows

In this example, the effect of the enzymatic composition according to the invention on the production performances of a herd of highly productive milk cows is described.

### Materials and Methods

38 highly productive milk cows of the Prim′Holstein breed were distributed into 2 groups and paired together. Their batching was carried out on the following criteria: milk production, protein level of the milk, fat level of the milk, lactation rank, live weight.

The animals received a mixed ration consisting of 9.5 kg of DM (dry material) of maize ensilage, 2.1 kg DM of fibrous feedstuff (straw+lucerne with long strands), 4.9 kg DM of nitrogen-containing corrector (the composition of which is described in Table 1), 8.0 kg of production feedstuff (the composition of which is described in Table 1).

The enzymatic composition, obtained by the method described in Example 1, was standardized as a premix for ensuring good homogeneity of the composition and facilitate its use during the step for manufacturing the feedstuffs. The thereby standardized premix was incorporated into the production feedstuff in an amount of 0.25% in order to provide 4,500 BGU/animal/day. The characteristics of the thereby standardized premix are: 130 AXC/g, 290 BGU/g and 35 CMC/g. The duration of the test is 8 weeks.

A supply of 1 kg/day of a feedstuff containing monopropyleneglycol (Sandi+, an energetic source) is achieved on cows with less than 30 days of lactation.

The average characteristics of the ration are (in %/dry matter): Total nitrogen-containing matter: 17.7%; Fibers insoluble in neutral detergents (NDF): 35.8% and Starch: 17.6%.

TABLE 1
Composition of the foodstuffs for milk cows

| | | | Production | |
|---|---|---|---|---|
| | | | feedstuff | |
| | | | (+enzymatic | |
| | Nitrogen- | Production | composition | |
| | containing | feedstuff | according to the | |
| Raw material (%) | corrector | (control) | invention) | Sandi+ |
| Rapeseed cake | 18.0 | | | |
| Expeller | | | | |
| Barley | 18.5 | 15.2 | 15.3 | 42.3 |
| Soya cake | 52.9 | 5.8 | 6.6 | |
| Rapeseed grain | | 7.8 | 7.9 | |
| Rapeseed cake | | 24.8 | 24.3 | 32.6 |
| Soya husk | | 20.0 | 20.0 | |
| Beet pulp | | 22.0 | 21.6 | |
| Straw | | 2.0 | 2.0 | |
| Bran | | | | 15.1 |
| Soybean oil | 1.7 | | | |
| Na bicarbonate | 1.3 | 1.5 | 1.5 | |
| Mineralized or | 3.9 | 0.3 | 0.3 | |
| vitamins | | | | |
| supplement (MVS) | | | | |
| Urea | 1.1 | | | |
| Premix | 0.5 | 0.5 | 0.5 | |
| Monopropylene | | | | 10.0 |
| glycol | | | | |
| Wheat | 2.3 | 0.3 | | |
| Enzymatic | | | 0.25 | |
| composition | | | | |
| according to the | | | | |
| invention | | | | |
| Dry matter (%) | 90.1 | 88.7 | 88.7 | 88.2 |
| Protein (% of | 35.0 | 18.0 | 18.0 | 18.0 |
| crude) | | | | |
| Fats (% of crude) | 5.0 | 5.1 | 5.1 | 2.2 |
| Crude cellulose | 6.4 | 16.4 | 16.4 | 8.0 |
| (%) | | | | |
| NDF (% of crude) | 16.4 | 35.0 | 35.0 | 24.0 |
| Insoluble fibers in | 9.2 | 21.1 | 21.0 | 11.0 |
| acid detergents | | | | |
| (ADF) (% of crude) | | | | |
| Lignin (ADL) (% of | 2.0 | 3.4 | 3.4 | 3.5 |
| crude) | | | | |
| UF milk (/kg crude) | 100.1 | 95.0 | 95.0 | 90.0 |
| PDIA (g/kg crude) | 116.1 | 55.7 | 56.0 | 48.6 |
| PDIN (g/kg crude) | 245.2 | 119.7 | 119.8 | 117.9 |
| PDIE (g/kg crude) | 164.3 | 106.5 | 106.9 | 97.5 |

The characteristics of the animal batches are described in Table 2.

TABLE 2
Characteristics of the batches of milk cows upon batching
(after 2 weeks before the experiment)

| | Control | Treatment |
|---|---|---|
| Number of animals | 19 | 19 |
| Lactation stage (d) | 168 | 185 |
| Milk production (kg/d) | 39.5 | 39.5 |
| Butyrous level (g/kg) | 35.9 | 35.9 |
| Protein level (g/kg) | 31.6 | 32.1 |
| Milk somatic cell (1,000/mL) | 391 | 220 |
| Urea (mg/L) | 267 | 284 |
| Live weight (kg) | 658 | 670 |

### Results

The production results of both groups are detailed in Table 3.

TABLE 3
Production results of the batches and statistics

| | | | Batch |
|---|---|---|---|
| | Control | Treatment | effect |
| Number of animals | 19 | 19 | |
| Milk production (MP) (kg/d) | 39.3 | 40.4 | *** |
| Butyrous level (BL) (g/kg) | 35.8 | 35.6 | NS |
| Protein level (PL) (g/kg) | 31.0 | 31.3 | ** |
| Exported Fat (g/d) | 1395 | 1413 | NS |
| Exported Protein (g/d) | 1210 | 1244 | *** |
| 7% Corrected milk production (kg/d) | 37.2 | 38.0 | ** |
| Milk somatic cell (1,000/mL) | 94 | 106 | NS |
| Live weight (kg) | 682 | 671 | *** |

NS: not significant;
*: p < 0.10;
**: p < 0.05;
***: p < 0.001

The inventors noticed that the provision of the enzymatic composition has a very pronounced effect on many criteria:
a significant increase in milk production;
significant increases in the protein level (+0.3 g/kg), and in the produced protein matter;
significant increase in MP 7% (milk production corrected by the levels=MP*(BL+PL)/70): +0.7 kg.

These effects are not accompanied by an increase in the ingested dry matter and therefore express a better food efficiency (amount of produced milk/ingested dry matter).

This example shows that providing an enzymatic composition according to the invention enables improving zootechnical performances of milk production and better valorization of the feed ration of the highly productive milk cows.

### Example 3

### Measurement of the Effect of the Enzymatic Composition According to the Invention on the Growth Performances of Bovine Animals for Fattening

In this example, the effect of the enzymatic composition according to the invention is described on the growth performances of bovine animals for fattening.

### Materials and Methods

40 bovine animals for fattening of the Charolais breed were distributed into two groups. The batching was carried out on the live weight criterion.

The animals received a mixed complete ration, the composition of which is detailed in Table 4.

The enzymatic composition, obtained by the method described in Example 1, was standardized as a premix in order to ensure good homogeneity of the composition and facilitate its use during the step of distribution to the animals. The thereby standardized premix was incorporated into the mixed complete ration in order to provide 9,000 BGU/animal/day. The characteristics of the thereby standardized premix are: 80 AXC/g, 150 BGU/g and 17 CMC/g.

The test lasted for 10 months.

TABLE 4
Composition of the mixed complete ration for bovine animals for fattening

| | Distributed amount (kg of crude/animal/day) | |
|---|---|---|
| | Fattening | Finishing |
| | from 300 to 500 kg of | from 500 to 750 kg |
| Ingredient | live weight | of live weight |
| Potato pulp | 3.0 | 3.0 |
| Wheat straw | 0.8 | 0.8 |
| Wrapped lucerne (33% of | 3.0 | 3.0 |
| dry matter) | | |
| Nitrogen-containing | 0.8 | 0.65 |
| concentrate (44% MAT) | | |
| Energetic concentrate | 2.5 | 3.5 |
| (1 UFV/kg) | | |
| Minerals | 0.1 | 0.1 |
| Urea | 0.01 | 0.06 |
| Maize silage and over- | 14.0 | 20.0 |
| pressed beets pulps (50/50 | | |
| of dry matter) | | |

### Results

The production results of both groups are detailed in table 5.

TABLE 5
Growth results of the batches

| | | Birth at T0 | | | | | |
|---|---|---|---|---|---|---|---|
| | | (8.3 | | T0 + 1 month | T0 + 2 months | | Carcass |
| | | months on | T0 to | to | to | T0 to | weight |
| DAWG (g/animal/day) | | average) | T0 + 1 month | T0 + 2 months | T0 + 8 months | T0 + 8 months | (kg) |
| Control | Average | 1 139 | 1 564 | 1 633 | 1 095 | 1 219 | 415 |
| | Standard | 139 | 369 | 340 | 236 | 186 | 30 |
| | deviation | | | | | | |
| Treatment | Average | 1 139 | 1 708 | 1 709 | 1 218 | 1 342 | 425 |
| | Standard | 117 | 590 | 293 | 197 | 200 | 39 |
| | deviation | | | | | | |

| | | | | | | |
|---|---|---|---|---|---|---|
| Improvement induced | 100 | 109 | 105 | 111 | 110 | 102 |
| by the treatment | (0%) | (9%) | (5%) | (11%) | (10%) | (2%) |
| expressed in base 100 | | | | | | |
| of the control | | | | | | |
| (% of improvement) | | | | | | |

The inventors noticed that by adding the enzymatic composition in the feed of young bovine animals for fattening, it was possible to improve by 10% the Daily Average Weight Gain (DAWG). This is expressed by a carcass weight upon slaughter, improved by 2% in the batch of animals supplemented with the enzymatic composition according to the invention.

This example shows that providing the enzymatic composition according to the invention enables improving the zootechnical performances of growth of bovine animals for fattening.

### Example 4

### Measurement of the Effect of the Enzymatic Composition According to the Invention on the Production Performances and the Rumination Activity of Milk Cows

In this example, the effect of the enzymatic composition according to the invention is described on the production performances of a herd of highly productive milk cows and on the rumination activity of the animals.

### Materials and Methods

36 highly productive milk cows of the Prim′Holstein breed were distributed into two groups. The batching of the animals was carried out on the criteria of milk production, live weight, lactation rank and lactation period in days. The test lasted for 85 days.

The animals received a mixed complete ration, mainly consisting of maize silage, Lucerne hay, rye-grass hay, corn gluten feed, production concentrate and water. The detailed composition is given in Table 6.

The enzymatic composition, prepared according to the production method described in Example 1, was standardized as a premix for ensuring good homogeneity of the composition and facilitating its use during the step of distribution to the animals (distribution as such, with [symbol] top feeding [symbol] ). The premix obtained was mixed in a total ration so as to provide 4,500 BGU/animal/day. The characteristics of the thereby standardized premix are: 400 AXC/g, 270 BGU/g and 69 CMC/g.

The rumination activity of the 36 cows was daily recorded with an acoustic detector (RuminAct, Milkline, Italy).

TABLE 6
Composition of the mixed complete ration of the milk cows

| | | |
|---|---|---|
| Maize silage | kg | 22.0 |
| Concentrate¹ + cotton cake | kg | 6.0 |
| Corn gluten meal-flaked barley mix. (60:40) | kg | 6.8 |
| Water | kg | 8.0 |
| Rye-grass hay | kg | 1.1 |
| Lucerne hay | kg | 4.4 |

¹soya cake 44%: 41.2%; cotton cake: 33%, sunflower cake: 7.21%; Corn gluten meal: 7.21%; CaCO₃: 2.68%; NaHCO₃: 2.68%; MgO: 1.00%; CaHPO₄: 0.67%; NaCl: 0.67%; ZnSO₄: 0.10%; Premix: 0.54%.

The nutritional values of the mixed complete ration were analyzed: dry matter 48.69%, crude proteins: 15.75%/dry matter (dry); Fats: 2.70%/dry matter; Crude cellulose: 18.45%/dry; NDF: 37.86%/dry; ADF: 21.15%/dry and starch: 25.57%/dry.

There was no ingestion difference between both batches during this test.

### Results

The milk production results of both groups are detailed in Table 7.

TABLE 7
Results of milk production (kg/animal/day) of the batches and statistics

| | | | | Effect of | Effect of | Interaction |
|---|---|---|---|---|---|---|
| | | | | the | the | (Treatment × |
| | | | Treatment | treatment | period | Period) |
| Periods | Control | Treatment | vs. Control | P = | P = | P = |
| D0-D21 | 36.7 ± 8.5 | 37.5 ± 8.4 | +0.8 | 0.7840 | 0.0001 | 0.9896 |
| D21-D37 | 36.9 ± 8.4 | 38.3 ± 8.6 | +1.4 | 0.6135 | 0.0001 | 0.4114 |
| D37-D53 | 35.6 ± 9.2 | 37.6 ± 8.1 | +2.0 | 0.4786 | 0.0001 | 0.1807 |
| D53-D69 | 34.1 ± 8.7 | 35.5 ± 8.1 | +1.4 | 0.6024 | 0.1024 | 0.9907 |
| D69-D85 | 33.7 ± 9.2 | 35.0 ± 8.0 | +1.3 | 0.6424 | 0.0001 | 0.9938 |
| D21-D85 | 35.1 ± 8.9 | 36.6 ± 8.3 | +1.5 | 0.5795 | 0.0001 | 0.9472 |

Milk production decreased during the test according to the lactation stage. During the test, the batch receiving the enzymatic composition according to the invention showed an improvement in milk production of +1.5 kg of milk per day and per animal as compared with the Control group.

The results of the milk composition (fats and proteins) are detailed in Table 8.

TABLE 8
Composition of the milk of the batches and statistics

| | | | | | |
|---|---|---|---|---|---|
| | | | | Treatment | |
| | | | Treatment | effect | Standard |
| Day | Control | Treatment | vs. Control | P = | error |

Butyrous level of the milk (%)

| | | | | | |
|---|---|---|---|---|---|
| D0 | 3.99 ± 0.52 | 3.87 ± 0.54 | −0.12 | 0.5102 | 0.1251 |
| D21 | 4.01 ± 0.74 | 4.00 ± 0.58 | −0.01 | 0.9421 | 0.1558 |
| D37 | 3.98 ± 0.49 | 4.02 ± 0.49 | +0.04 | 0.7988 | 0.1162 |
| D53 | 4.12 ± 0.73 | 4.34 ± 0.61 | +0.22 | 0.3368 | 0.1581 |
| D69 | 4.15 ± 0.54 | 4.30 ± 0.69 | +0.15 | 0.4860 | 0.1462 |
| D85 | 4.09 ± 0.62 | 4.21 ± 0.55 | +0.12 | 0.5305 | 0.1376 |

Production of milk fats (kg/day)

| | | | | | |
|---|---|---|---|---|---|
| D0 | 1.42 ± 0.33 | 1.38 ± 0.29 | −0.04 | 0.7318 | 0.0731 |
| D21 | 1.42 ± 0.25 | 1.47 ± 0.31 | +0.05 | 0.5751 | 0.0662 |
| D37 | 1.44 ± 0.23 | 1.51 ± 0.27 | +0.07 | 0.4430 | 0.0585 |
| D53 | 1.38 ± 0.29 | 1.51 ± 0.33 | +0.13 | 0.2094 | 0.0729 |
| D69 | 1.37 ± 0.31 | 1.48 ± 0.26 | +0.11 | 0.2739 | 0.0675 |
| D85 | 1.26 ± 0.23 | 1.34 ± 0.22 | +0.08 | 0.2790 | 0.0535 |

Protein level of the milk (%)

| | | | | | |
|---|---|---|---|---|---|
| JD0 | 3.57 ± 0.40 | 3.46 ± 0.31 | −0.11 | 0.3732 | 0.0844 |
| D21 | 3.60 ± 0.45 | 3.78 ± 0.22 | +0.18 | 0.1243 | 0.0832 |
| D37 | 3.66 ± 0.32 | 3.80 ± 0.43 | +0.14 | 0.2724 | 0.0887 |
| D53 | 3.66 ± 0.33 | 3.87 ± 0.22 | +0.21 | 0.0324 | 0.0671 |
| D69 | 3.67 ± 0.30 | 3.84 ± 0.23 | +0.17 | 0.0659 | 0.0624 |
| D85 | 3.69 ± 0.31 | 3.82 ± 0.31 | +0.13 | 0.2092 | 0.0727 |

Production of milk proteins (kg/day)

| | | | | | |
|---|---|---|---|---|---|
| D0 | 1.26 ± 0.19 | 1.24 ± 0.22 | −0.02 | 0.7599 | 0.0490 |
| D21 | 1.29 ± 0.23 | 1.42 ± 0.35 | +0.13 | 0.2103 | 0.0705 |
| D37 | 1.34 ± 0.30 | 1.41 ± 0.19 | +0.07 | 0.4245 | 0.0590 |
| D53 | 1.24 ± 0.27 | 1.37 ± 0.35 | +0.13 | 0.2293 | 0.0729 |
| D69 | 1.23 ± 0.32 | 1.34 ± 0.29 | +0.11 | 0.2692 | 0.0708 |
| D85 | 1.15 ± 0.253 | 1.23 ± 0.24 | +0.08 | 0.3567 | 0.0579 |

During the test, the butyrous level remained stable in the Control group while it increased in the Treatment group (supplemented with the enzymatic composition according to the invention). The butyrous level was always higher in the batch of treated cows than in the batch of the control cows. After 85 days of test, the cows of the Treatment group had a butyrous level greater by 0.12 point to that of the cows of the Control group.

During the test, the protein level of the milk increased faster in the Treatment group than in the Control group. Statistically, the inventors noticed a significant increase in the protein level at D53 and at D69, by +0.21 and +0.17 points respectively. This is also expressed by a greater export of proteins (+0.08 kg/day at the end of the test).

The rumination activity is of great importance for the metabolic activity of milk cows and may be a useful tool for monitoring animal health. Rumination stimulates production of saliva and therefore ensures optimum conditions for the cellulolytic activity in the rumen.

The average daily rumination activity data are given in Table 9. The rumination activity is expressed in the number of minutes of rumination per day and per animal.

TABLE 9
Rumination activity of the animals (minutes of rumination/animal/day)

| | | | | | | Interaction |
|---|---|---|---|---|---|---|
| | | | | Effect of the | Effect of | (Treatment × |
| | | | Treatment | treatment | the period | Period) |
| Periods | Control | Treatment | vs. Control | P = | P = | P = |
| D0-D21 | 500.6 | 511.6 | +11.0 | 0.6513 | 0.0001 | 0.4360 |
| D21-D37 | 497.0 | 515.8 | +18.8 | 0.4730 | 0.0001 | 0.6990 |
| D37-D53 | 458.3 | 497.8 | +39.5 | 0.1858 | 0.0001 | 0.2840 |
| D53-D69 | 482.3 | 528.1 | +45.9 | 0.1210 | 0.0322 | 0.3747 |
| D69-D85 | 471.7 | 523.9 | +52.2 | 0.1019 | 0.0001 | 0.0132 |
| D21-D85 | 477.3 | 516.4 | +39.1 | 0.1657 | 0.0001 | 0.0066 |

Both groups do not show any statistical difference at the beginning of the test (500.6 vs. 511.6 min/day/animal for the Control and Treatment groups respectively). Nevertheless, after 16 days of testing, the rumination activity numerically increases in the Treatment batch. In the last period of the test (D69-D85), the interaction (treatment×period) is significant (P=0.0132) with a rumination activity of the Treatment group greater than that of the Control group (+52.2 minutes/day/animal). Statistical analysis over the period D21-D85 shows the same interaction effect with +39 min/day/animal of rumination activity in cows of the Treatment group.

This example demonstrates that providing the enzymatic composition according to the invention enables improving the zootechnical performances of milk production (daily milk production, production of milk proteins and of milk fats), better valorization of the feed ration of highly productive milk cows and an improved rumination activity.

### Example 5

### Measurement of the Effect of the Enzymatic Composition According to the Invention on Ruminal Fermentations In Vitro (Gas Release)

In this example the effect of the enzymatic composition according to the invention is described on ruminal fermentations in vitro, in particular the releases of gas and notably the production of methane.

### Materials and Methods

Animals

Two culled cows with cannulas to the rumen were used. During the whole of the duration of the experiment in vitro, the animals were fed with a mixture of haylage grass (4 kg), maize silage (5 kg/d), chopped straw (1 kg) and concentrate (1 kg).

Ingredients

Four feedstuffs were studied in vitro: Grass hay, Grass silage, Maize silage and soya cake. The chemical composition of the experimental ingredients is shown in Table 10.

TABLE 10
Chemical composition of the ingredients used

| | | | | Crude energy |
|---|---|---|---|---|
| Ingredients | MAT¹ (%) | NDF (%) | Starch (%) | (kcal/kg DM) |
| Soya | 55.37 | 15.18 | N/A² | 4636.92 |
| Haylage | 17.90 | 44.72 | N/A | 4106.45 |
| Maize | 5.07 | 53.53 | 19.5 | 4546.62 |
| Hay | 6.89 | 65.93 | N/A | 4306.68 |

¹MAT: Total nitrogen-containing material
²N/A: Not applicable

The different ingredients were freeze-dried and ground to 1 mm by means of a laboratory grinder.

The enzymatic composition prepared according to the production method described in Example 1 by using a strain of Aspergillus neoniger, was standardized as a premix in order to ensure good homogeneity of the composition and facilitate its use during the step of distribution to the animals in [symbol] top feeding [symbol] . The obtained premix was mixed in the total ration so as to provide 3,375 BGU/animal/day. The characteristics of the thereby standardized premix are: 70 AXC/g, 213 BGU/g and 28 CMC/g.

The fermentations in vitro were achieved according to the method described by Menke and Steingass (1988). The inoculum consisted for ⅓ of rumen fluid collected on 2 cannulated cows and ⅔ of a Menke and Steingass buffer (Menke K H, Steingass H. (1988) Estimation of the energetic feed value obtained from chemical analysis and in vitro gas production using rumen fluid. Animal Research Development. 28, 7-55). 200 mg of substrate were incorporated in 100 ml glass syringes in the presence of the inoculum. The read out of the gas volume was performed regularly.

The rumen fluid was sampled on animals subjected to a normal feed diet (Control), and then on the same animals after 3 weeks of adaptation to a diet supplemented with the enzymatic composition according to the invention (Test) in an amount of 1 g/kg of foodstuff (˜15 g/d/animal).

The experimental scheme was the following: 4 substrates×2 treatments (Control or Test)×3 syringes+3 blanks=27 syringes per series. Two series were carried out (so 54 syringes) for determining the production of methane (CH₄) and of ammoniacal nitrogen (N—NH₃).

The fermentations of the series dedicated to the production of CH₄ and N—NH₃ were carried out for 24 h. At the end of the 24 h, a sample of the gas phase of the fermentation bottles was sampled in order to determine the production of CH₄ by gas chromatography and a sample of the liquid phase for determining the production of N-NH₃. Upon starting the fermentations, 3 samples of the inoculum were also centrifuged and analyzed for their N—NH₃ content.

The CH₄ productions were related to the g of DM of substrate set to ferment and the values were corrected according to the inocula and to the blanks.

### Results

From the point of view of the amount of methane produced after 24 h of fermentation, there seems to appear a tendency for producing less methane with the enzymatic composition according to the invention for the fodder ingredients (hay, haylage and maize silage) and a greater use of ammonia by the ruminal microbiot (lower NH₃ content), as shown by Table 11.

TABLE 11
Production of methane and ammonium after fermentation in vitro for
24 h of feedstuffs with ruminal fluid either in the presence or not of
the enzymatic composition according to the invention (N = 6)

| | | | change/ | | |
|---|---|---|---|---|---|
| | | CH₄ | control | NH₃ | change/ |
| | | (mmol/g) | (%) | (mg/g) | control (%) |
| Hay | control | 1.779 | | 27.50 | |
| | Test | 1.623 | −8.8 | 26.90 | −2.2 |
| Maize silage | control | 2.293 | | 21.40 | |
| | Test | 2.274 | −0.8 | 20.70 | −3.3 |
| Grass silage | control | 1.428 | | 28.90 | |
| | Test | 1.391 | −2.6 | 25.80 | −10.7 |
| Soya cake | control | 2.315 | | 67.70 | |
| | Test | 2.513 | 8.6 | 68.00 | 0.4 |
| SEM | | 0.075 | | 2.90 | |

Upon measurements conducted after 24 h (methane and NH₃), the inventors were able to observe a consistent tendency for all the fiber feedstuffs (fodders: hay, maize silage and grass haylage) with reduction of the production of methane and with a greater use of the ammonia of the fermentation buffer by the micro-organisms of the rumen. This means that in the first hours of fermentation, the fodders incubated in presence of the enzymatic composition according to the invention may promote better bacterial growth and consequently ensure better supply of proteins and amino acids of microbial origin to the cows.

This example shows that by providing the enzymatic composition according to the invention enables reducing the production of methane by the micro-organisms contained in the rumen of the ruminants.

### Example 6

### Measurement of the Effect of the Enzymatic Composition According to the Invention on the Digestibilities in Sacco of Feedstuffs for Ruminants

In this example the effect of the enzymatic composition according to the invention is described on the degradability in sacco of various common raw materials in the feed of the ruminants.

### Materials and Methods

Animals

Two culled cows cannulated to the rumen were used. During the whole duration of the experiment in sacco, the animals were fed with a mixture of haylage grass (4 kg), maize silage (5 kg/j), chopped straw (1 kg) and concentrate (1 kg).

Ingredients

Four feedstuffs were studied in sacco: Grass hay, Hay silage, Maize silage and Soya cake. The chemical composition of the experimental ingredients is shown in Table 12.

TABLE 12
Chemical composition of the ingredients used

| | | | | Crude energy |
|---|---|---|---|---|
| Ingredients | MAT¹ (%) | NDF (%) | Starch (%) | (kcal/kg DM) |
| Soya | 55.37 | 15.18 | N/A² | 4636.92 |
| Haylage | 17.90 | 44.72 | N/A | 4106.45 |
| Maize | 5.07 | 53.53 | 19.5 | 4546.62 |
| Hay | 6.89 | 65.93 | N/A | 4306.68 |

¹MAT: Total nitrogen-containing material
²N/A: Not applicable

The different ingredients were freeze-dried and ground to 1 mm by means of a laboratory grinder.

The enzymatic composition prepared according to the production method described in Example 1 by using a strain of Aspergillus neoniger, was standardized as a premix for ensuring good homogeneity of the composition and facilitating its use during the step for distribution to the animals as [symbol] top feeding [symbol] . The obtained premix was mixed in the total ration so as to provide 3,375 BGU/animal/day. The characteristics of the thereby standardized premix are: 70 AXC/g, 213 BGU/g and 28 CMC/g.

In order to study the changes induced by the enzymatic composition according to the invention on the ruminal metabolism in the animal, the 2 cannulated dry dairy cows were used for conducting an experiment in sacco with the use of the technique with nylon bags (Ørskov, E. R., Hovell, D and Mould, F. L. (1980). The use of the nylon bag technique for the evaluation of feedstuffs. Tropical Animal Production 5: 195-213.). Two series of digestibilities in sacco were performed on the four tested feedstuffs: a series with the animals subject to the feed diet described above (Control), followed by a series performed on the same animals after 3 weeks of adaptation to a supplemented diet (Test) with the enzymatic composition according to the invention in an amount of about 1 g/kg of feedstuff (˜15 g/d/animal). The experimental scheme was the following: 4 ingredients×2 treatments (Control or Test)×4 bags×7 measurement times (0, 3, 6, 9, 12, 24, 48 h)=224 bags. The fermented feedstuffs in the bags (0, 6, 12, 24, 48 h) were analyzed for their crude protein contents, NDF, energy and starch contents (starch exclusively for the maize silage).

The dry matter and crude proteins contents of the ingredients before and after hydrolysis were determined via reference methods (method 967.03; method 981.10; “Official methods of analysis”, AOAC, 1990, Ed. Kenneth Helrich). The starch contents of the maize silage were determined by an enzymatic and colorimetric method (Megazyme Ltd, Ireland). The NDF (Neutral Detergent Fiber) contents according to the method of Van Soest et al. (Van Soest P. J., Robertson J. B., Lewis B. A. (1991): Methods for dietary fiber, neutral detergent fiber, and nonstarch polysaccharides in relation to animal nutrition. J. Dairy Sci., 74, 3583-3597) were determined by using thermostable amylase with the ANKOM system.

At the end of the digestions in sacco, the digestibilities of the dry matter, of the proteins, of the energy, of the NDF and of the starch were calculated and the values statistically compared according to the relevant ingredient and either with supplementation of not (N=8) and the degradation kinetics modeled by means of the model of Orskov and McDonald (Orskov, E. R. and McDonald, Y. 1979. Protein degradability in the rumen was estimated from determining the digestibility of feeds in the rumen. (Daynal Agricultural Science, Cambridge 92:499-503). The cow was considered as a random factor.

### Results

The final degradability of the dry matter of the ingredients contained in the nylon bags incubated in the rumen of the cannulated cows was not significantly altered by the distribution of the enzymatic composition according to the invention to the cows (Table 13). However, the degradation kinetics was actually influenced for the fodders and in particular as regards the haylage grass since the degradability after 24 h reached 0.820 in the presence of the enzymatic composition according to the invention versus 0.645 without it (P<0.05).

This tendency was confirmed in a more rapid degradation of the fraction of insoluble fibers in the neutral detergent (NDF) (Table 14). Indeed, the addition of the enzymatic composition according to the invention makes the degradability of the NDF after 24 h to pass from 0.370 to 0.685 for haylage grass (P<0.05), and the same tendency is observed for maize and hay, as well as for nitrogen (Table 15) with however non-significant differences (P>0.05).

The degradability of the crude energy contained in the soya after 6 and 12 h was greater when the cows were supplemented with the enzymatic composition according to the invention as compared with the non-supplemented cows (0.600 and 0.540 vs. 0.420 and 0.490) (Table 16).

TABLE 13
Digestibility of the dry material in sacco in the presence or not
of the enzymatic composition according to the invention (N = 4)
(g/g of feedstuff) after 0, 3, 6, 9, 12, 24 and 48 h

| | | Digestibility of the dry matter | | | | | | |
|---|---|---|---|---|---|---|---|---|
| | | (g/g of feedstuff, n = 4) | | | | | | |
| | | Fermentation time (h) | | | | | | |
| | | 0 | 3 | 6 | 9 | 12 | 24 | 48 |
| Soya | Test | 0.409 | 0.480 | 0.625 | 0.695 | 0.715 | 0.880 | 0.968 |
| | Control | 0.409 | 0.496 | 0.480 | 0.651 | 0.735 | 0.860 | 1.001 |
| Grass silage | Test | 0.409 | 0.424 | 0.495 | 0.551 | 0.560 | 0.820 | 0.855 |
| | Control | 0.409 | 0.444 | 0.470 | 0.540 | 0.590 | 0.645 | 0.845 |
| Maize silage | Test | 0.407 | 0.477 | 0.515 | 0.489 | 0.605 | 0.650 | 0.760 |
| | Control | 0.407 | 0.474 | 0.480 | 0.534 | 0.545 | 0.585 | 0.735 |
| Hay | Test | 0.235 | 0.242 | 0.270 | 0.307 | 0.320 | 0.445 | 0.570 |
| | Control | 0.235 | 0.263 | 0.290 | 0.316 | 0.315 | 0.390 | 0.550 |

In Bold: significant difference (p <0.05)

TABLE 14
Digestibility of NDF in sacco in the presence or not of the enzymatic
composition according to the invention (N = 2) (g/g of feedstuff)
after 0, 6, 12, 24 and 48 h.

| | | Digestibility of the NDF (g/g of feedstuff, | | | | |
|---|---|---|---|---|---|---|
| | | n = 2) | | | | |
| | | Fermentation time (h) | | | | |
| | | 0 | 6 | 12 | 24 | 48 |
| Soya | Test | 0.242 | 0.390 | 0.480 | * | * |
| | Control | 0.240 | 0.270 | 0.605 | * | * |
| Grass silage | Test | 0.028 | 0.080 | 0.270 | 0.685 | 0.765 |
| | Control | 0.028 | 0.060 | 0.200 | 0.370 | 0.730 |
| Maize silage | Test | 0.072 | 0.225 | 0.360 | 0.440 | 0.620 |
| | Control | 0.072 | 0.180 | 0.285 | 0.335 | 0.585 |
| Hay | Test | 0.028 | 0.045 | 0.120 | 0.285 | 0.455 |
| | Control | 0.028 | 0.100 | 0.125 | 0.220 | 0.430 |

*: not enough sample in the nylon bag for carrying out the assay
In Bold: Significant Difference (P <0.05)

TABLE 15
Digestibility of the nitrogen in sacco in the presence or not of the
enzymatic composition according to the invention (N = 2)
(g/g of feedstuff) after 0, 6, 12, 24 and 48 h.

Digestibility of the MAT (g/g of feedstuff,
n = 2)
Fermentation time (h)

| | | 0 | 6 | 12 | 24 | 48 |
|---|---|---|---|---|---|---|
| Soya | Test | 0.228 | 0.590 | 0.595 | * | * |
| | Control | 0.228 | 0.325 | 0.625 | * | * |
| Grass silage | Test | 0.303 | 0.665 | 0.720 | 0.910 | 0.955 |
| | Control | 0.303 | 0.640 | 0.740 | 0.785 | 0.935 |
| Maize silage | Test | 0.705 | 0.705 | 0.745 | 0.765 | 0.780 |
| | Control | 0.705 | 0.715 | 0.705 | 0.745 | 0.780 |
| Hay | Test | 0.303 | 0.420 | 0.395 | 0.525 | 0.655 |
| | Control | 0.303 | 0.375 | 0.415 | 0.475 | 0.655 |

*: not enough sample in the nylon bag for carrying out the assay
In Bold Significant Difference (P <0.05)

TABLE 16
Digestibility of the crude energy in sacco in the presence or not of the
enzymatic composition according to the invention (N = 2)
(g/g of feedstuff) after 0, 6, 12, 24 and 48 h.

| | | Digestibility of the crude energy | | | | |
|---|---|---|---|---|---|---|
| | | (g/g of feedstuff, n = 2) | | | | |
| | | Fermentation time (h) | | | | |
| | | 0 | 6 | 12 | 24 | 48 |
| Soya | Test | 0.374 | 0.600 | 0.540 | * | * |
| | Control | 0.374 | 0.420 | 0.490 | * | * |
| Grass silage | Test | 0.199 | 0.435 | 0.660 | 0.806 | 0.838 |
| | Control | 0.199 | 0.415 | 0.505 | 0.678 | 0.827 |
| Maize silage | Test | 0.414 | 0.520 | 0.530 | 0.570 | 0.760 |
| | Control | 0.414 | 0.490 | 0.550 | 0.590 | 0.680 |
| Hay | Test | 0.199 | 0.240 | 0.285 | 0.415 | 0.490 |
| | Control | 0.199 | 0.220 | 0.285 | 0.350 | 0.515 |

*: not enough sample in the nylon bag for carrying out the assay
In Bold: Significant Difference (P <0.05)

The hypothesis of a significant impact of the enzymatic composition according to the invention on the fiber feedstuffs is confirmed with these in sacco degradability measurements. The enzymatic composition according to the invention accelerates the degradation processes of the 3 fiber feedstuffs and significantly it allows acceleration of the fermentation of haylage grass and particularly of its fiber fraction (NDF). The consequences in milk cows might be a reduction in the ruminal bulkiness and an increase in the capability of ingesting fodders. This effect would be of particular interest for lactation animals which have difficulties in satisfying their needs for food by consuming fodders and which consequently have to be supplemented in a significant way by more expensive production concentrates than the fodder resources.

This example demonstrates that providing the enzymatic composition according to the invention enables a better valorization of the fodder resources by the animals and therefore a better food efficiency.

### Example 7

### Measurement of the Effect of the Enzymatic Composition According to the Invention on the Metabolism of Microbes of the Rumen in an Anaerobic Reactor with Overflow (OARs) and in Culture Tubes

The goals of this example are:

1) To quantify the effects of the enzymatic composition according to the invention on the metabolism of microbes of the bovine rumen cultivated in 6 overflow anaerobic reactors (OAR) on three characteristic rations of bovine livestock. The OARs are continuous fermenters with double effluent.

2) To specify the mode of action of the enzymatic composition according to the invention on the ruminal fermentations, by measuring, in culture tubes, the impact of the crude enzymatic composition, of its soluble and insoluble fractions and of the enzymatic composition inactivated by autoclaving on the productions of volatile fatty acids (VFA) and of gas after 24 h of incubation.

### Materials and Methods

Test in an Overflow Anaerobic Reactor (OAR)

Six fermenters with a working volume of 1 litre are used, adjusted to the renewal rates of the solid phase and of the liquid phase of 0.03 and 0.06 h⁻¹ respectively. These fermenters are inoculated with the contents of the milk cow rumen filtered on a 1 mm web. The pH of the fermenters is maintained above 6.0 by continuous infusion of buffer solutions (carbonate and phosphate). The supply of substrates (feedstuffs) is carried out at a set time: at 11 h and 23 h. A phase of equilibration of the fermenters is carried out during the first 5 days before the phase of measurements and samplings.

The enzymatic composition according to the invention is introduced at a level of 10 g per kg of DM ration. The daily supply is 0.25 g per fermenter. Two enzymatic composition batches are prepared according to the production method described in Example 1 by using a strain of Aspergillus tubingensis (no. 1) on the one hand and a strain of Aspergillus neoniger (no. 2) on the other hand. The thereby obtained enzymatic compositions have the following features: for version no. 1 1,037 AXC/g, 1,063 BGU/g and 262 CMC/g; for version no. 2 3,399 AXC/g, 3,623 BGU/g and 846 CMC/g. Both obtained compositions were standardized as two premixes in order to facilitate its incorporation into the fermenters. The characteristics of the thereby standardized premixes are: 171 AXC/g, 225 BGU/g and 67 CMC/g for version no. 1; 122 AXC/g, 280 BGU/g and 75 CMC/g for version no. 2.

The rations contain wheat and a soya cake so as to formulate iso-nitrogenous and iso-energetic diets.

The various tested factors are:

1) Product, factor at 3 levels: absence/enzymatic composition no. 1/enzymatic composition no. 2

2) Diets, factor at 3 levels: based on maize silage/grass silage/orchard grass hay

The selected experimental plan is a plan in 3 balanced incomplete blocks (Table 17) corresponding to 3 experimental periods of 7 days, each comprising a 5-day equilibration phase and a phase of measurements and samplings of 2 days.

TABLE 17
Experimental plan of the tests in OARs with two versions of enzymatic
composition and the various feedstuffs.

| | | fermenters | | | | | |
|---|---|---|---|---|---|---|---|
| | | A | B | C | D | E | F |
| block 1 | Diet | EH | EM | F | F | EM | EH |
| | Enzymatic | No. 1 | No. 1 | 0 | No. 2 | 0 | No. 2 |
| | composition | | | | | | |
| block 2 | Diet | F | EH | EH | EM | F | EM |
| | Enzymatic | No. 2 | 0 | No. 1 | No. 2 | No. 1 | 0 |
| | composition | | | | | | |
| block 3 | Diet | EH | EM | EM | F | EH | F |
| | Enzymatic | 0 | No. 1 | No. 2 | 0 | No. 2 | No. 1 |
| | composition | | | | | | |

EH: grass silage;
EM: maize silage;
F: prairie hay

The measurements and samplings carried out during the tests are:
pH, redox potential.
fermenter contents for counting protozoa and determination of the fermentation facies (VFA, ammoniacal nitrogen).
Gas (collected for 2 days) for measuring the produced volume and its composition
Effluents collected for two days, for degradation results of the rations, fermentation and synthesis of microbial proteins.
Microbial pellets by double centrifugation of the fresh effluents.

The following analyses were conducted on the samples:
VFA (chromatography) in silages, effluents and the fermenter contents
Ammoniacal nitrogen (specific probe)
Gas (chromatography)
DM, organic material in the feedstuffs and the freeze-dried effluents
Starch in the feedstuffs and the freeze-dried effluents
Fractionation of the walls according to Van Soest (NDF, ADF, ADL) in the feedstuffs and freeze-dried effluents
Nitrogen (Dumas method) in the feedstuffs, the freeze-dried effluents and the microbial pellets.
Nucleic bases (chromatography) in the freeze-dried effluents and the microbial pellets.

A statistical analysis is carried out with a variance analysis model as follows: Yᵢⱼₖₗ=Pᵢ+Rⱼ+Pᵢ*Rⱼ+Bₖ+eᵢⱼₖₗ with P, R, B and e being the Product and Diet factors, the block and the experimental error, respectively.

Test in Culture Tubes

60 culture tubes of 72 mL fitted with plugs with a butyl-teflon septum are inoculated with the contents of the fermenter sampled 12 h after the last supply of ration and incubated for 24 h according to the standard procedure. The studied factors are:

1) Product, P factor at 5 levels: absence/Composition no. 2/insoluble fraction of Composition no. 2/liquid fraction of Composition no. 2 (without the fermentation substrate)/Enzymatic composition inactivated by autoclaving. The liquid fraction of Composition no. 2 corresponds to the enzymes extracted in an aqueous phase from the remainder of the Composition no. 2.

2) Inoculum, R factor at 3 levels: adapted to a diet based on maize silage/grass silage/dactyl hay and receiving the same diet in culture tubes.

The enzymatic composition according to the invention no. 2 is introduced with the ration at a level of 10 g per kg of DM ration.

The experimental plan is organized in 2 periods of 2 consecutive days for each inoculum. The inocula are sampled on the OARs not receiving the enzymatic composition (level 0 of the Product factor in the experimental plan in OARs) after the equilibration period. Two culture tubes are initiated per treatment (Product×Inoculum combination) and per day of incubation.

The measurements and samplings carried out during the tests are:
Inoculum for determining its VFA composition
Culture medium after 24 h of incubation for tubes for determining the VFA composition.
Gas for measuring the produced amount after 24 h of incubation and determination of its composition.

The following analyses are conducted on the samplings:
VFA (chromatography) in silages, effluents and fermenter contents
Gas (chromatography)

A statistical analysis is conducted with a variance analysis model.

This analysis shows the beneficial effect of the enzymatic composition according to the invention on the metabolism of the microbes of the rumen, as compared with the effect of the isolated enzymes of the fermentation substrate on this metabolism.

### Example 8

### Production of an Enzymatic Composition According to the Invention by Solid-State Fermentation

In this example is described a method for obtaining the enzymatic composition according to the invention by solid-state fermentation of a substrate consisting of a mixture of rapeseed cake and wheat bran (main substrate) and of maize germ cake (additional substrate) in the presence of a strain of Aspergillus tubingensis.

A nutritive medium is formed with a mixture of rapeseed cake as a flour and wheat bran completed with a maize germ cake, in the following weight proportion: 36% of rapeseed cake, 36% of wheat bran and 28% of maize germ cake. The mixture is then pre-humidified to 60% of dry matter and autoclaved for 35 min at 105° C. After cooling, the medium is inoculated with a solution of spores of Aspergillus tubingensis in order to obtain a concentration of 1×10⁷ spores per gram of dry matter and an initial humidity of 45%. The pH is adjusted to 4.9 by adding sulfuric acid. The thereby obtained culture medium is distributed in Erlenmeyer vials in an amount of 10 g of dry matter per vial. The Erlenmeyer vials are then incubated at 33° C. under aerobic conditions in darkness for 72 h without any stirring. The culture is stopped upon occurrence of the first spores in the culture medium (since the presence of spores may bother the animals during ingestion because of their volatility, the fermented products are designed so as to contain as little as possible of them).

The obtained mixture at the end of the fermentation may be dried and used as such in the feed of the animals or be extracted in water so as to be added in liquid form.

The characteristics of the thereby obtained composition after 72 h of fermentation are: 2,650 AXC/g, 4,808 BGU/g and 1,050 CMC/g.

1. An enzymatic composition comprising a main substrate, selected from the group consisting of wheat, wheat bran, rapeseed cake, a mixture of wheat and wheat bran, a mixture of wheat and rapeseed cake, a mixture of wheat bran and rapeseed cake, and a mixture of wheat, wheat bran and a rapeseed cake, fermented with a strain of Aspergillus section Nigri, said enzymatic composition displaying:
(i) a xylanase activity greater than or equal to 500 AXC per gram of composition,
(ii) a β-glucanase activity greater than or equal to 500 BGU per gram of composition, and
(iii) a cellulase activity greater than or equal to 50 CMC per gram of composition.

2. The enzymatic composition according to claim 1, wherein the strain of Aspergillus section Nigri is a strain of the clade Aspergillus niger.

3. The enzymatic composition according to claim 1, wherein the strain of Aspergillus section Nigri is a strain of the sub-clade Aspergillus tubingensis.

4. The enzymatic composition according to claim 1, wherein the strain of Aspergillus section Nigri is a strain of Aspergillus tubingensis or Aspergillus neoniger.

5. The enzymatic composition according to claim 1, wherein the strain of Aspergillus section Nigri is a strain of Aspergillus tubingensis.

6. The enzymatic composition according to claim 1, wherein the substrate is fermented with the strain of Aspergillus section Nigri by solid-state fermentation.

7. The enzymatic composition according to claim 1, wherein the substrate consists of a mixture of wheat bran and rapeseed cake.

8. The enzymatic composition according to claim 7, wherein the wheat bran and the rapeseed cake are in mass proportions ranging from 20/80 to 50/50.

9. The enzymatic composition according to claim 1, displaying:
(i) a xylanase activity greater than or equal to 1000 AXC per gram of composition,
(ii) a β-glucanase activity greater than or equal to 800 BGU per gram of composition, and
(iii) a cellulase activity greater than or equal to 120 CMC per gram of composition.

10. A method for manufacturing an enzymatic composition as defined in claim 3, comprising a step of solid-state fermentation of a main substrate, selected from the group consisting of wheat, wheat bran, rapeseed cake, a mixture of wheat and wheat bran, a mixture of wheat and rapeseed cake, a mixture of wheat bran and rapeseed cake, and a mixture of wheat, wheat bran and rapeseed cake, with a strain of the sub-clade A. tubingensis, at least until the fermentation product displays the following minimum values of enzymatic activity:
(i) a xylanase activity greater than or equal to 500 AXC per gram of composition,
(ii) a β-glucanase activity greater than or equal to 500 BGU per gram of composition, and
(iii) a cellulase activity greater than or equal to 50 CMC per gram of composition.

11. The method for manufacturing according to claim 10, wherein the strain of the sub-clade Aspergillus tubingensis is a strain of Aspergillus tubingensis or of Aspergillus neoniger.

12. The method for manufacturing according to claim 11, wherein the strain of the sub-clade Aspergillus tubingensis is a strain of Aspergillus tubingensis.

13. An additive for the feed of ruminants which comprises the enzymatic composition according to claim 1.

14. (canceled)

15. A premix for the feed of ruminants comprising an additive according to claim 13.

16. A supplemented feed composition for ruminants comprising an effective amount of an additive according to claim 13 or of a premix according to claim 15, in association with feedstuffs suitable for ruminants.

17. A method for manufacturing a supplemented feed composition for ruminants comprising mixing an additive according to claim 13 with feedstuffs suitable for ruminants.

18-19. (canceled)

20. A method for improving zootechnical performances of a ruminant in which the ruminant is made to ingest an effective amount of the enzymatic composition according to claim 1.

21. The method according to claim 20, which is for increasing milk production and/or improving the consumption index and/or the food efficiency and/or promoting weight gain and/or increasing the rumination activity and/or increasing the digestibility of the fodders and concentrates and/or reducing the production of methane.

22. A method for manufacturing a supplemented feed composition for ruminants comprising mixing a premix according to claim 15 with feedstuffs suitable for ruminants.

23. A method for improving zootechnical performances of a ruminant in which the ruminant is made to ingest an effective amount of the additive according to claim 13.

24. A method for improving zootechnical performances of a ruminant in which the ruminant is made to ingest an effective amount of the premix according to claim 15.

25. A method for improving zootechnical performances of a ruminant in which the ruminant is made to ingest an effective amount of the feed composition according to claim 16.
